(19) United States
(12) Patent Application Publication
(10) Pub. No.: US 2020/0321117 A1
(43) Pub. Date: Oct. 8, 2020

(54) SYSTEMS AND METHODS FOR ASSESSING SIGNS AND SYMPTOMS IN CONGESTIVE HEART FAILURE PATIENTS

(71) Applicant: International Business Machines Corporation, Armonk, NY (US)

(72) Inventors: Uri Kartoun, Cambridge, MA (US); Kenney Ng, Arlington, MA (US); Tanya Rudakevych, Boston, MA (US); Charalambos Stavropoulos, Hastings-on-Hudson, NY (US); Sophie Batchelder, Lancaster, PA (US); Veronica Aldous, Davie, FL (US); Amy Chiu, San Francisco, CA (US); David Osofsky, Arlington, MA (US); Francis Campion, Westwood, MA (US); Paul C. Tang, Los Altos, CA (US)

(21) Appl. No.: 16/375,407

(22) Filed: Apr. 4, 2019

(51) Int. Cl. G16H 50/20; G16H 50/70; G06N 20/00; G06K 9/62; G16H 10/60

(52) U.S. Cl. CPC G16H 50/20; G16H 50/70; G16H 10/60; G06K 9/6267; G06N 20/00

(57) ABSTRACT

A system and associated method provides an assessment of patient's heart health status based on past medical data. The system has a collection system, a processing system, a profiling system, and a user tools system. The collection system collects data from at least one data source having the past medical data of a plurality of patients. The processing system processes unstructured data to extract information related to patient health at different points in time. The profiling system develops a classifier based on the extracted information and other collected data. The profiling system further categorizes the extracted information into one of a plurality of health statuses at the different points in time. The user tools system generates a user interface comprising the plurality of health statuses at the different points in time. The health statuses relate to a compensated or decompensated status of the patient's heart.

### TECHNICAL FIELD

The present application relates to assessing signs and symptoms of a patient and, more particularly, to providing physicians with a comprehensive tool for assessing the progression and status of a disease such as congestive heart failure (CHF) in a patient based on a universe of available data for the patient and similarly-situated patients.

### BACKGROUND

CHF is an end-stage life chronic condition that affects millions of Americans every year. After pneumonia, CHF is the second most common cause of hospital admission. The financial burdens associated with CHF care are immense; in 2004, the 1.1 million U.S. admissions for CHF amounted to nearly $29 billion in hospital charges.

In addition to frequent admissions that characterize CHF patients, such patients often meet their primary care physician in the outpatient setting. In such outpatient encounters (also called “office visits”) the physician attempts to assess the level of severity of the patient. Then the physician can adjust the patient's disease management strategy. For instance, the physician may change a dose of a current medication, add a new medication, or stop a current medication. The assessment is based on manually observing the number and severity of signs and symptoms characterizing the patient's disease improvement or deterioration, as well as relying on additional criteria such as laboratory values (e.g., B-type natriuretic peptide [BNP]) and weight increase or decrease. The physician may thus rely on their own expertise and knowledge and past information regarding the patient and similar patients within medical records. This approach is useful, but limited. The physician cannot practically consider and synthesize all available data for CHF patients, especially when the dataset is large and the past medical records do not follow a particular pattern or format, as is often the case.

Some tools have been developed to standardize available data for CHF patients to provide a patient profile, such as that described in U.S. Pat. No. 6,572,557, entitled “System and Method for Monitoring Progression of Cardiac Disease State Using Physiologic Sensors” (“the '557 Patent”). In the '557 Patent, “monitoring is implemented by ongoing surrogate measurement of standard and direct measurements, such as daily activity and respiratory and cardiac response rate, utilizing existing implantable, rate-responsive stimulation devices that incorporate activity, respiration, and/or other sensors.” While this and other similar approaches may be useful, they are limited in their ability to use existing structured and unstructured data to provide a specialized tool for assisting a physician in assessing the status of a patient and determining treatment. The '557 Patent, for instance, relies on specific test measurement results to assess the status. This solution requires a change in the way that data is collected and does not account for data that may already be available using historical medical record-keeping, including clinical narrative notes. Therefore, there is a need for a system that is capable of using existing medical record data and data collection techniques to provide an assessment tool for CHF patients.

The present disclosure is directed to overcoming these and other problems of the prior art.

### SUMMARY

In some embodiments, a computer-implemented method for assessing patient health in a data processing system is disclosed. The data processing system includes a processing device and a memory comprising instructions which are executed by the processor to perform the method. The method includes collecting data from at least one data source, the at least one data source including past medical information for a plurality of patients, and training a machine learning classifier to categorize data elements as being one of a plurality of health statuses based on the past medical information for the plurality of patients. The method further includes categorizing past medical data related to a particular patient at different points in time into one of the plurality of health statuses, based on the classifier, and generating a user interface having the health status of the particular patient at each point in time, based on the categorization.

In other embodiments, a data analysis system for providing user tools associated with assessing a health status of a patient is disclosed. The data analysis system includes a collection system, a processing system, a profiling system, and a user tools system. The collection system collects data from at least one data source including past medical information for a particular patient. The past medical information includes a combination of structured and unstructured data. The processing system processes the unstructured data to extract information related to patient health at different points in time. The profiling system categorizes the combination of structured data with the extracted information related to patient health into one of a plurality of health statuses at the different points in time. The user tools system generates a user interface comprising the plurality of health statuses at the different points in time.

In other embodiments, a computer-implemented method for assessing a health status of a patient's heart in a data processing system is disclosed. The data processing system includes a processing device and a memory comprising instructions which are executed by the processor to perform the method. The method includes collecting data from at least one data source, the at least one data source including past medical information for a particular patient, the past medical information comprising clinical narrative notes describing a health of the particular patient's heart as well as structured data such as laboratory values, medications prescribed, vital signs, and procedures. The method further includes performing natural language processing of the clinical narrative notes to extract information indicating the health of the patient's heart at different points in time. The method also includes categorizing the extracted information into either a compensated or a decompensated health status of the patient's heart at the different points in time, and generating a user interface comprising the compensated or decompensated health statuses at the different points in time.

### BRIEF DESCRIPTION OF THE DRAWINGS

The foregoing and other aspects of the present invention are best understood from the following detailed description when read in connection with the accompanying drawings. For the purpose of illustrating the invention, there is shown in the drawings embodiments that are presently preferred, it being understood, however, that the invention is not limited to the specific instrumentalities disclosed. Included in the drawings are the following Figures:

FIG. 1 is a block diagram of an exemplary healthcare data environment, consistent with disclosed embodiments;

FIG. 2 is a block diagram of an example data processing system in which aspects of the illustrative embodiments are implemented;

FIG. 3 is a block diagram of an exemplary data analysis system, consistent with disclosed embodiments;

FIG. 4 is a block diagram of an exemplary data source, consistent with disclosed embodiments;

FIG. 5 is a flowchart of an exemplary process for assessing a health of a patient, consistent with disclosed embodiments;

FIG. 6 is a listing of example clinical narrative notes associated with a compensated status of a patient's heart;

FIG. 7 is a listing of example clinical narrative notes associated with a decompensated status of a patient's heart; and

FIG. 8 is a timeline visualization of a health status of a patient's heart at different periods in time.

### DETAILED DESCRIPTION OF EXEMPLARY EMBODIMENTS

Embodiments of the present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a head disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network (LAN), a wide area network (WAN) and/or a wireless network. The network may comprise copper transmission cables, optical transmission fibers, wireless transmission, routers, firewalls, switches, gateway computers, and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object-oriented programming language such as Java, Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer, or entirely on the remote computer or server. In the latter scenario, the remote computer may be connected to the user's computer through any type of network, including LAN or WAN, or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operations steps to be performed on the computer, other programmable apparatus, or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical functions. In some alternative implementations, the functions noted in the block may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The present disclosure relates to a system and method capable of analyzing the historical and current medical record of a CHF patient to more comprehensively assist a physician in assessing the level of severity of the patient's heart condition. By using natural language processing techniques enhanced with machine learning algorithms, the system is capable of providing the physician with an automated and interactive view of the patient's current and historical disease status. In some embodiments, the system is configured to present the physician with a timeline view of the patient's heart status using existing medical record data for the patient and other similarly-situated patients.

The disclosed system, in at least some embodiments, includes a data analysis system that collects information from data sources. The data sources include medical records for a plurality of patients, as well as medical variables, such as diagnosis and treatment data. The medical records may include structured data (such as labs, comorbidities, demographics) as well as unstructured data (clinical narrative notes, discharge summaries, radiology and pathology reports). The data analysis system extracts informative descriptors related to the patient's heart from the data sources.

Often the patient's heart condition is documented in a very recent interaction with the care system (e.g., the patient has just been discharged and came to see his primary care physician at his office a few days after discharge). In other cases the patient's historical clinical narrative notes (including all inpatient and outpatient) are informative even if they indicate relatively-old historical events (e.g., an admission occurred 2 years prior to the visit). A disclosed analysis system is configured to process all of these data elements and present a trajectory of improvement and/or deterioration of the patient's heart status to the physician, providing a useful visualization of the patient's heart over time and improving decision support capabilities.

In some embodiments, the system analyzes a historical record of the patient using natural language processing and machine learning methods to determine a patient's heart status at any point in time. A desirable heart status may be denoted as “compensated” and a deteriorating status may be denoted as “decompensated.” The system is capable of calculating probabilities for compensated and decompensated statuses at any time over the patient's history. The system may implement user tools to assist a physician in understanding the review of the patient's medical records. For example, the system may present a timeline and trajectory of compensation and decompensation statuses for each date and time the patient interacted with the healthcare system. The user tools may provide the physician with a look at the associations between medical variables and heart condition, and thus the physician may be able to provide more effective care to a patient going forward. For instance, the physician may be able to more easily identify the medical variable (e.g., the medication, condition, illness, etc.) that may be contributing to the patient's health status.

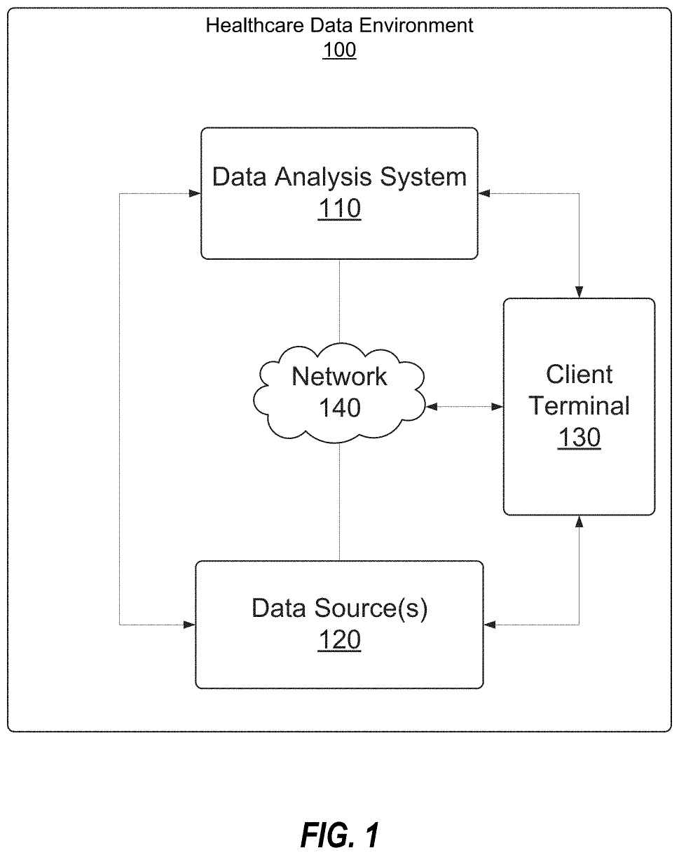

FIG. 1 is an illustration of an exemplary healthcare data environment 100. The healthcare data environment 100 may include a data analysis system 110, one or more data sources 120, and a client terminal 130. A network 140 may connect the data analysis system 110, the one or more data sources 120, and/or the client terminal 130.

The data analysis system 110 may be a computing device, such as a back-end server. The data analysis system 110 may include components that enable data assessment functions for providing one or more tools to a physician (or to other clinicians such as a nurse) based on analysis of past medical data. The one or more data sources 120 may be computing devices and/or storage devices configured to supply data to the data analysis system 110. For example, the one or more data sources 120 may include a computing device having healthcare data collection software associated with patient records, medical devices, etc. Examples of healthcare data that may be supplied by the one or more data sources 120 include, for example, patient visit data (e.g., number of visits, date of visit, reason for visit, location, time, length of visit, etc.), testing data (tests completed, result values etc.), clinical narrative notes (e.g., data input by a medical professional during a patient visit), administrative information, information captured while the patient's location is at home or travelling captured by commercially available wearable devices in real-time such as wireless heart failure monitor systems, etc. The client terminal 130 may be a computing device, such as an end-user device (e.g., a desktop or laptop computer, mobile device, etc.). The client terminal 130 may communicate with the data analysis system 110 in order to receive and produce an interactive user interface that is presented to a user.

The network 140 may be a local or global network and may include wired and/or wireless components and functionality which enable internal and/or external communication for components of the healthcare data environment. The network 140 may be embodied by the Internet, provided at least in part via cloud services, and/or may include one or more communication devices or systems which enable data transfer to and from the systems and components of the healthcare data environment 100.

In accordance with some exemplary embodiments, the data analysis system 110, data source(s) 120, client terminal 130, or the related components include logic implemented in specialized hardware, software executed on hardware, or any combination of specialized hardware and software executed on hardware, for implementing the healthcare data environment 100 or related components. In some exemplary embodiments, the data analysis system 110 or any of its components may be or include the IBM Watson™ system available from International Business Machines Corporation of Armonk, N.Y., which is augmented with the mechanisms of the illustrative embodiments described hereafter.

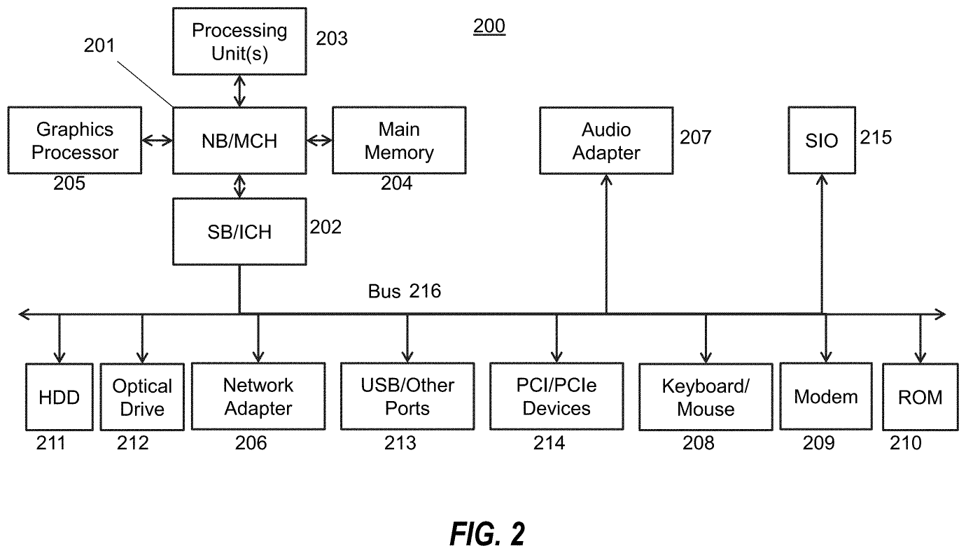

FIG. 2 is a block diagram of an example data processing system 200 in which aspects of the illustrative embodiments are implemented. Data processing system 200 is an example of a computer in which computer usable code or instructions implementing the process for illustrative embodiments of the present invention are located. In one embodiment, the data processing system 200 represents one or more of the data analysis system 110, the one or more data sources 120, or the client terminal 130, and implements at least some of the functional aspects described herein.

In the depicted example, data processing system 200 can employ a hub architecture including a north bridge and memory controller hub (NB/MCH) 201 and south bridge and input/output (I/O) controller hub (SB/ICH) 202. Processing unit 203, main memory 204, and graphics processor 205 can be connected to the NB/MCH 201. Graphics processor 205 can be connected to the NB/MCH 201 through an accelerated graphics port (AGP).

In the depicted example, the network adapter 206 connects to the SB/ICH 202. The audio adapter 207, keyboard and mouse adapter 208, modem 209, read only memory (ROM) 210, hard disk drive (HDD) 211, optical drive (CD or DVD) 212, universal serial bus (USB) ports and other communication ports 213, and the PCI/PCIe devices 214 can connect to the SB/ICH 202 through bus system 216. PCI/PCIe devices 214 may include Ethernet adapters, add-in cards, and PC cards for notebook computers. ROM 210 may be, for example, a flash basic input/output system (BIOS). The HDD 211 and optical drive 212 can use an integrated drive electronics (IDE) or serial advanced technology attachment (SATA) interface. The super I/O (SIO) device 215 can be connected to the SB/ICH 202.

An operating system can run on processing unit 203. The operating system can coordinate and provide control of various components within the data processing system 200. As a client, the operating system can be a commercially available operating system. An object-oriented programming system, such as the Java™ programming system, may run in conjunction with the operating system and provide calls to the operating system from the object-oriented programs or applications executing on the data processing system 200. As a server, the data processing system 200 can be an IBM® eServer™ System p® running the Advanced Interactive Executive operating system or the Linux operating system. The data processing system 200 can be a symmetric multiprocessor (SMP) system that can include a plurality of processors in the processing unit 203. Alternatively, a single processor system may be employed.

Instructions for the operating system, the object-oriented programming system, and applications or programs are located on storage devices, such as the HDD 211, and are loaded into the main memory 204 for execution by the processing unit 203. The processes for embodiments of the website navigation system can be performed by the processing unit 203 using computer usable program code, which can be located in a memory such as, for example, main memory 204, ROM 210, or in one or more peripheral devices.

A bus system 216 can be comprised of one or more busses. The bus system 216 can be implemented using any type of communication fabric or architecture that can provide for a transfer of data between different components or devices attached to the fabric or architecture. A communication unit such as the modem 209 or network adapter 206 can include one or more devices that can be used to transmit and receive data.

Those of ordinary skill in the art will appreciate that the hardware depicted in FIG. 2 may vary depending on the implementation. For example, the data processing system 200 includes several components which would not be directly included in some embodiments of the data analysis system 110, data source(s) 120, or client terminal 130.

Moreover, other internal hardware or peripheral devices, such as flash memory, equivalent non-volatile memory, or optical disk drives may be used in addition to or in place of the hardware depicted. Moreover, the data processing system 200 can take the form of any of a number of different data processing systems, including but not limited to, client computing devices, server computing devices, tablet computers, laptop computers, telephone or other communication devices, personal digital assistants, and the like. Essentially, data processing system 200 can be any known or later developed data processing system without architectural limitation.

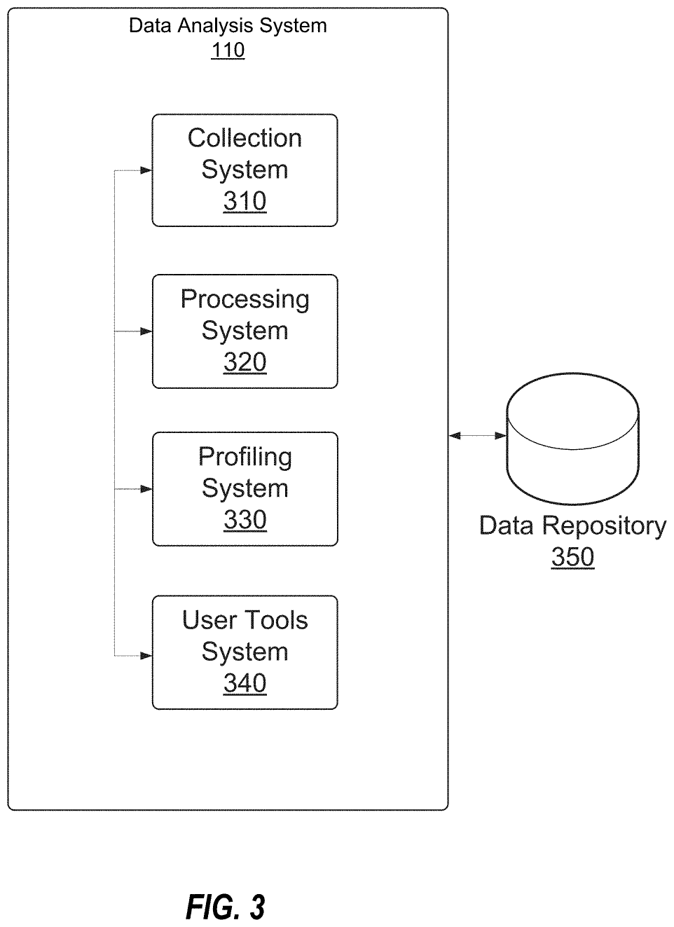

FIG. 3 illustrates an exemplary embodiment of the data analysis system 110. In an exemplary embodiment, the data analysis system 110 includes a collection system 310, a processing system 320, a profiling system 330, and a user tools system 340. These subsystems of the data analysis system 110 may be components of a single device, or may be separated devices connected to each other (e.g., via the network 140). In some embodiments, the data analysis system 110 may further include and/or be connected to a data repository 350.

The collection system 310 may be a computing device or component (e.g., software or hardware engine or module) configured to collect data from the one or more data sources 120. The collection system 310 is an intake point of the data analysis system 110. The collection system 310 receives data and is configured to route the data throughout the data analysis system 110 to enable the functions of the data analysis system 110. The collection system 310 performs filtering, categorization, and/or scoring of the data to allow for efficient processing. The collection system 310 may also collect current medical status information, such as information provided at a current physician visit.

The processing system 320 may be a computing device or component (e.g., software or hardware engine or module) configured to process data collected by the collection system 310 to review, categorize, classify, and extrapolate data. For example, the processing system 320 performs natural language processing of clinical narrative notes to identify terms and phrases that are relevant to a medical status of the patient at a given point in time. The processing system 320 may categorize data based on a date of treatment or visit.

The profiling system 330 may be a computing device or component (e.g., software or hardware engine or module) configured to create a patient profile based on the processed data from the processing system 320 and other data received by the collection system 310. The profiling system 330 may be configured to develop a classifier using machine learning trained on the collected medical data associated with a plurality of patients of similar conditions. The profiling system 330 may use supervised or unsupervised learning techniques and ground truth to train the classifier.

The profiling system 330 may be further configured to use the classifier to categorize a health status of a particular patient at a given point in time (e.g., the time that coincides with a doctor visit or procedure). For instance, the profiling system 330 may determine whether a patient's heart was compensated or decompensated at each data point (e.g., patient visit, treatment date, etc.) based on the classifier.

The user tools system 340 may be a computing device or component (e.g., software or hardware engine or module) configured to provide one or more user tools to the client terminal 130 based on the output of the profiling system 330. For example, the user tools system 340 may provide a timeline tool that presents the decompensated/compensated status of the patient over time. The user tools system 340 may be configured to combine the timeline with additional medical variables (e.g., medications, procedures, etc.) that may provide insight to a physician regarding possible causes and/or effects of the patient's health status (e.g., decompensated/compensated status). In another example, the user tools system 340 may provide a medical decision support system that utilizes the patient profile data to provide recommendations for patient treatment based on current medical status information.

The data repository 350 may be a database configured to store data. The data repository 350 may be configured to receive data from the collection system 310 and/or from one or more data sources 120 and store the data according to appropriate storage protocols. In some embodiments, the data repository 350 receives data from the data analysis system 110, such as from the collection system 310. In other embodiments, the data repository 350 receives data from the one or more data sources 120 and is a data supply for the data analysis system 110. In some embodiments, the data repository 350 may store one or more patient profiles having information generated by the profiling system 330 based on the data collected by the collection system 310 and processed by the processing system 320.

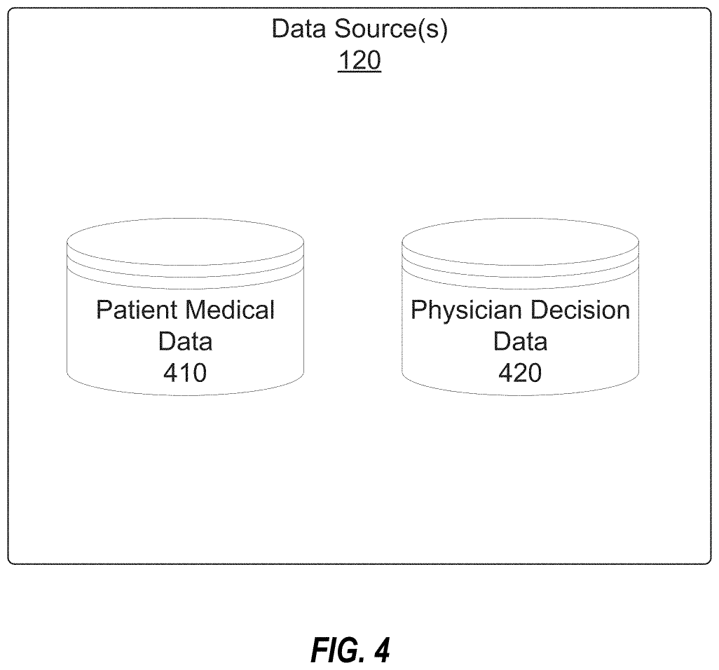

FIG. 4 is a block diagram of an example of the one or more data sources 120. In one example, the data source(s) 120 include a patient medical data storage element 410 and a physician decision data storage element 420. While illustrated as separate storage elements, it should be understood that the data may be stored together or separately in different configurations. In an exemplary embodiment, the medical data storage element 410 stores a collection of data related to patients having CHF, the data including information such as demographics, allergies, diagnoses, vital signs, laboratory tests, operations, providers, physicals, pathology reports, clinical narrative notes, discharge summaries, radiology reports, cardiology reports, and encounters. The physician decision data storage element 420 stores a collection of data related to possible physician decisions surrounding treatment options for CHF patients. In one example, the physician decision data storage element 420 stores procedures and medications that may be prescribed for a CHF patient, as well as detailed information regarding the procedures and medications (such as effectiveness, side effects, risk factors, etc.).

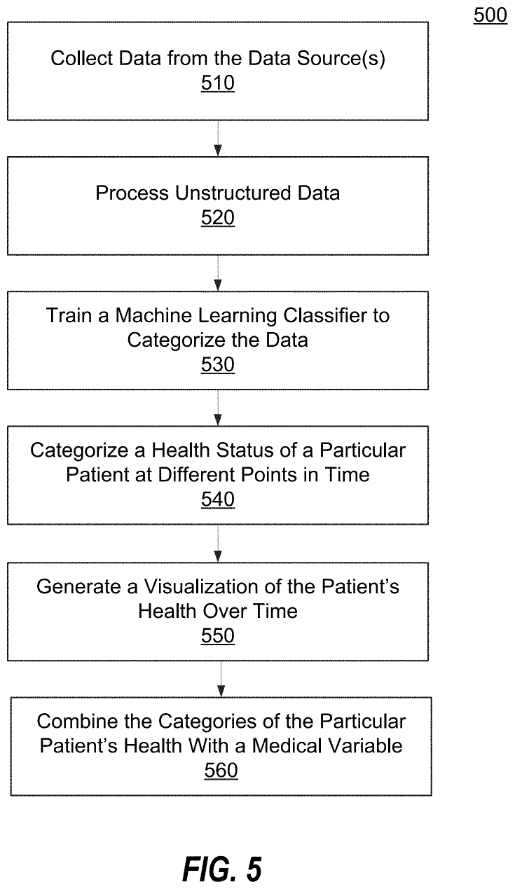

FIG. 5 is a flowchart of an exemplary process for providing a clinical tool for assessing heart health status for a CHF patient. The data analysis system 110 performs one or more steps of the process 500 in order to use information from data source(s) 120 to produce a tool for assisting in an assessment of the health of a patient, specifically the patient's heart health at the time of an evaluation. While the process 500 is described in relation to heart health status, it should be understood that the concepts may be applied to other health status determinations (such as chronic obstructive pulmonary disease [COPD] or asthma).

In step 510, the collection system 310 collects data from the data source(s) 120. This step may include the collection system 310 receiving data directly from the one or more data source(s) 120 and storing the raw data in the data repository 350. In another embodiment, the collection system 310 may receive data from the data repository 350.

The collection system 310 may collect data from the patient medical data storage element 410. The data from the patient medical data storage element 410 may include structured data and unstructured data. The structured data may be already categorized and/or in a format that is usable by the data analysis system 110. An example of structured data may include lab results, comorbidities, or patient demographics. The unstructured data may be uncategorized and/or in a format that needs further processing by the data analysis system 110. An example of unstructured data may include clinical narrative notes. Clinical narrative notes may include notations made by a practitioner during a patient office visit or admission. Clinical narrative notes could be in any format, depending on the standards, practices, and habits of the practitioner that created them. Both the structured and unstructured data may relate to a set of patients with the same or similar condition, such as patients with CHF.

In step 520, the processing system 320 processes the unstructured data. For example, the processing system 320 performs natural language processing to extract information from the clinical narrative notes. The processing system 320 may use other techniques to extract information from the unstructured or structured data. Data processing applied to notes may include a variety of text extraction methods. For example, it may include “Text Nailing”, a method that relies on brute-force and human in-the-loop steps. This method is described, for example, in “Text nailing” Kartoun U.; ACM Interactions(2017). It may also include methods such as described in “Extracting Information From Textual Documents in the Electronic Health Record: A Review of Recent Research” Meystre, S. M; Savova, G. K, Kipper-Schuler, K. C, Hurdle, J. F; Yearbook of Medical Informatics (2008), and “Clinical Information Extraction Applications: A Literature Review” Wang, Yanshan; Wang, Liwei; Rastegar-Mojarad, Majid; Moon, Sungrim; Shen, Feichen; Afzal, Naveed; Liu, Sijia; Zeng, Yuqun; Mehrabi, Saeed; Sohn, Sunghwan; Liu, Hongfang; Journal of Biomedical Informatics (2018).

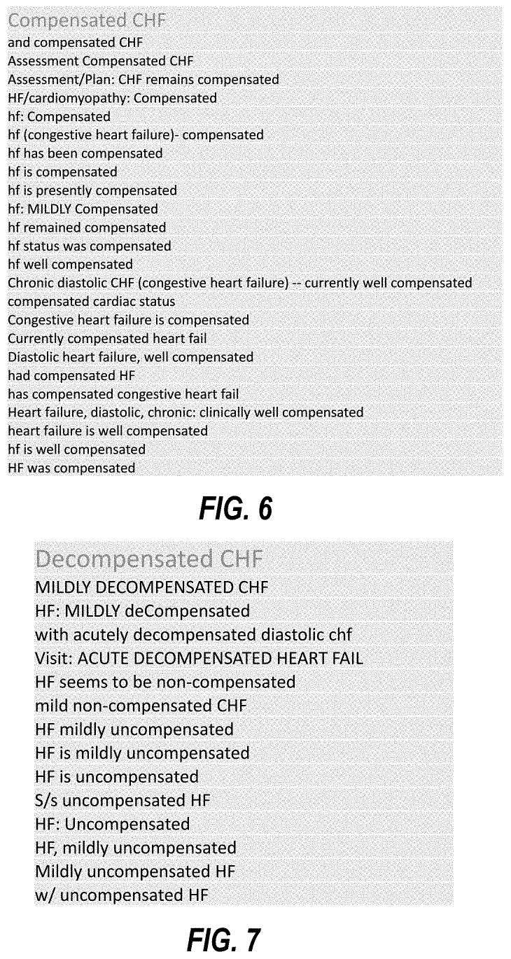

In step 530, the profiling system 330 trains a machine learning classifier to categorize the structured and unstructured data as they relate to the health of a patient's heart. For example, the profiling system 330 may be supplied with a set of known parameters that relate to different conditions. FIGS. 6 and 7 provide examples of data elements that may be used as ground truth for training a machine learning classifier to distinguish clinical narrative notes as being associated with “compensated” or “decompensated” status of the patient's heart. The profiling system 330 may use supervised learning to improve the ground truth and the accuracy of the classifier. In one embodiment the classifier is trained to identify if a single clinical narrative note is associated with a compensated or a decompensated heart status. A training set composed of collection of notes of CHF patients is used to train a machine learning algorithm. Each note in the training set is labeled as “compensated” or “decompensated” by a domain expert (typically a physician). The notes and their associated labels serve as a “ground truth” (or “gold standard”) indicating the highest confidence level of the status of each such note. Once the training set is created (notes and labels) a text extraction mechanism extracts variables representing each note. Variables may include measurements (e.g., ejection fraction: 56%) or textual (e.g., “ejection fraction: normal,” “heart failure: decompensated”). A large collection of such variables may represent each note and its associated label. Once the training set is created it is used by a machine learning algorithm to identify associations between the variables and the label of each note. The profiling system 330 generates an equation or multiple equations indicating the level of association between any given new note and its calculated label. In some instances, the profiling system 330 calculates a probability for a note to belong to each class. The training set could be enhanced by additional variables not directly related to the specific note, for example, by lab values extracted on the date of the note recorded or recent, and additional variables found in the medical databases, such as the patient's age, gender, comorbidities, and medications. A machine leaning classifier trained with this enhanced set of data could be used to more accurately calculate the probability for the patient to be considered “compensated” or “decompensated” in any given date and time.

In step 540, the profiling system 330 categorizes a health status of the patient at different points in the past based on the processed data. For example, the profiling system 330 may use the machine learning classifier to choose between a plurality of categories that describe the patient's health at a time associated with data. For example, the profiling system 330 may classify the patient as “healthy” or “stable” based on data associated with an office visit that occurred two years ago (e.g., due to normal test results, clinical narrative notes that indicate that the patient is healthy, etc.). The profiling system 330 may classify the patient as “unhealthy” or “not in baseline” at the time of another office visit (e.g., due to abnormal test results or clinical narrative notes indicating a deterioration of the patient's heart functionality).

In an exemplary embodiment, the profiling system 330 is configured to categorize the patient's heart as “compensated” or “decompensated” at various points in time. In this example, the patient may be a CHF patient. The profiling system 330 may use a machine learning classifier that uses ground truth that particularly identifies data elements as either being associated with “compensated” or “decompensated” status of the patient's heart.

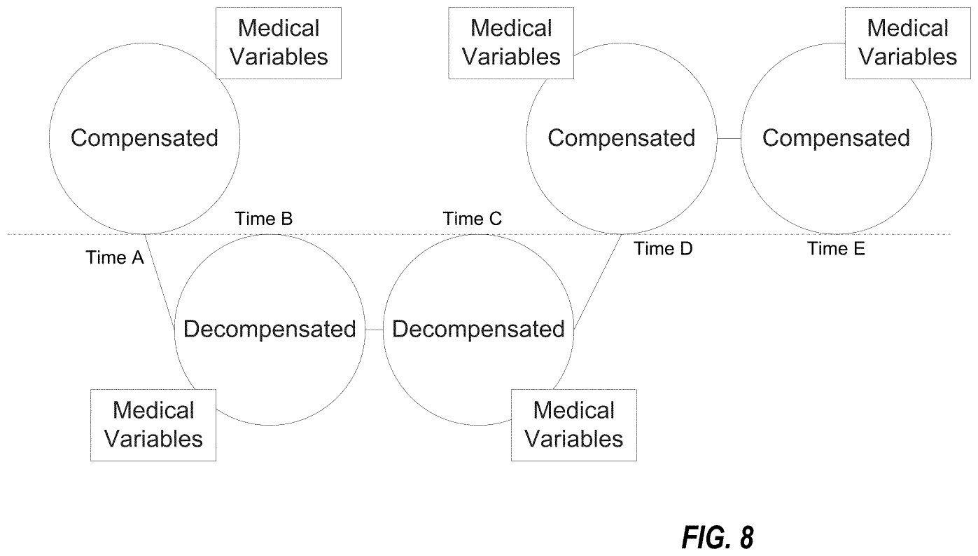

In step 550, the user tools system 340 generates a visualization of the patient's health over time based on the classifications made by the profiling system 330. FIG. 8 is an example of a visualization that identifies the compensated/decompensated statuses of the patient's heart over time. The visualization may include a timeline of the health status over time. The user tools system 340 may generate a user interface of the visualization and transmit the user interface to the client terminal 130 for displaying to a user.

In step 560, the user tools system 340 combines the categorization of the patient's health at different points in time with other information to provide a tool for assessing a current status of the patient's health. For instance, the user tools system 340 may combine the timeline with information from the physician decision data storage element 420. In an example, the information from the physician decision data storage element 420 includes a medical variable, such as procedures, medications, illnesses, dietary advice, and the like. For example, the user tools system 340 may associate procedures and medications that occurred around the time of each health classification with the health classification. For instance, if the patient is determined to have a “compensated” heart health at time A, the user tools system 340 would associate the procedures and medications that may explain the “compensated” classification. These associations may be added to the visualization, such as that shown in FIG. 8.

In some embodiments, the process 500 may further include the user tools system 340 providing a recommendation based on current health data received by the collection system 310. For example, the user tools system 340 may communicate with the profiling system 330 to determine a compensated or decompensated status of the patient's heart. In another example, the user tools system 340 may analyze the visualization and/or categorized health status of the patient and provide a recommendation for a course of action (e.g., procedure, medication, etc.). For example, the user tools system 340 may include or work in combination with a medical decision support system to provide a recommendation based on categorization of the health status of the patient.

As a result of the process 500, the client terminal 130 is provided with a clinical tool that can be used to assess the health of a patient and make decisions regarding a suggested course of action. The data analysis system 110 thus provides a solution for the difficulty of assessing the health status of a CHF patient's heart (or other similar health scenarios). The data analysis 110 implements a practical application of stored medical records data in order to determine the health status of a patient over time and provide useful tools to a physician (or other clinical user) for providing effective care. Where previous systems may rely on manual review of a patient's medical history and personal experience of a medical team, the disclosed data analysis system is capable of reviewing large data sets related to medical records for a plurality of patients. The system may then use the discovered associations identified in the data to create an automated classifier for determining the health status of an individual patient at different points in time. The system thus provides an innovative tool for assessing the health of a patient as it varies over time.

The disclosed systems and methods also provide an advantage in that they use existing past medical data, which may be structured or unstructured. In this way, the disclosed system can rely on information from sources such as clinical narrative notes, which often have the physician's most direct statement of a health of the patient. Prior art systems rely on constant monitoring or specifically-structured data in order to produce a visualization of the patient's health over time.

The present description and claims may make use of the terms “a,” “at least one of,” and “one or more of,” with regard to particular features and elements of the illustrative embodiments. It should be appreciated that these terms and phrases are intended to state that there is at least one of the particular feature or element present in the particular illustrative embodiment, but that more than one can also be present. That is, these terms/phrases are not intended to limit the description or claims to a single feature/element being present or require that a plurality of such features/elements be present. To the contrary, these terms/phrases only require at least a single feature/element with the possibility of a plurality of such features/elements being within the scope of the description and claims.

In addition, it should be appreciated that the following description uses a plurality of various examples for various elements of the illustrative embodiments to further illustrate example implementations of the illustrative embodiments and to aid in the understanding of the mechanisms of the illustrative embodiments. These examples are intended to be non-limiting and are not exhaustive of the various possibilities for implementing the mechanisms of the illustrative embodiments. It will be apparent to those of ordinary skill in the art in view of the present description that there are many other alternative implementations for these various elements that may be utilized in addition to, or in replacement of, the example provided herein without departing from the spirit and scope of the present invention.

The system and processes of the Figures are not exclusive. Other systems, processes and menus may be derived in accordance with the principles of embodiments described herein to accomplish the same objectives. It is to be understood that the embodiments and variations shown and described herein are for illustration purposes only. Modifications to the current design may be implemented by those skilled in the art, without departing from the scope of the embodiments. As described herein, the various systems, subsystems, agents, managers, and processes can be implemented using hardware components, software components, and/or combinations thereof. No claim element herein is to be construed under the provisions of 35 U.S.C. 112, sixth paragraph, unless the element is expressly recited using the phrase “means for.”

Although the invention has been described with reference to exemplary embodiments, it is not limited thereto. Those skilled in the art will appreciate that numerous changes and modifications may be made to the preferred embodiments of the invention and that such changes and modifications may be made without departing from the true spirit of the invention. It is therefore intended that the appended claims be construed to cover all such equivalent variations as fall within the true spirit and scope of the invention.

What is claimed is:

1. A computer-implemented method for assessing the health of a patient in a data processing system comprising a processing device and a memory comprising instructions which are executed by the processor, the method comprising:
collecting data from at least one data source, the at least one data source including past medical information for a plurality of patients;
training a machine learning classifier to categorize data elements as being one of a plurality of health statuses based on the past medical information for the plurality of patients;
categorizing past medical data related to a particular patient at different points in time into one of the plurality of health statuses, based on the classifier; and
generating a user interface having the health status of the particular patient at each point in time, based on the categorization.

2. The method of claim 1, wherein the past medical information for a plurality of patients includes structured data and unstructured data.

3. The method of claim 2, wherein the unstructured data includes clinical narrative notes.

4. The method of claim 3, wherein the method further comprises performing natural language processing of the clinical narrative notes to extract information.

5. The method of claim 1, wherein the user interface comprises a timeline of the health statuses of the particular patient.

6. The method of claim 1, further comprising associating the health status of the particular patient at each point in time with a medical variable.

7. The method of claim 6, further comprising combining the timeline and the associated medical variables.

8. The method of claim 6, wherein the medical variable comprises a medication or medical procedure associated with the particular patient.

9. The method of claim 1, wherein the plurality of health statuses comprise a health status of a patient's heart.

10. The method of claim 9, wherein the health status of the patient's heart is categorized as decompensated or compensated.

11. The method of claim 1, wherein the at least data source comprises a medical records database.

12. The method of claim 1, further comprising:
receiving current health data for the particular patient;
categorizing a current health status of the particular patient based on the current health data and the classifier; and
generating a user interface comprising the current health status of the particular patient.

13. The method of claim 12, wherein the current health status relates to a health of the patient's heart, and the health status is categorized as either compensated or decompensated.

14. A data analysis system for providing user tools associated with assessing a health status of a patient, comprising:
a collection system that collects data from at least one data source, the at least one data source including past medical information for a particular patient, the past medical information comprising a combination of unstructured data and structured data related to patient health;
a processing system that processes the unstructured data to extract information related to patient health at different points in time;
a profiling system that categorizes the structured data and extracted information related to patient health into one of a plurality of health statuses at the different points in time;
a user tools system that generates a user interface comprising the plurality of health statuses at the different points in time.

15. The data analysis system of claim 14, wherein the unstructured data comprises clinical narrative notes.

16. The data analysis system of claim 15, wherein the processing system performs natural language processing of the clinical narrative notes to extract the information related to patient health at different points in time.

17. The data analysis system of claim 14, wherein the user interface includes a visualization of the plurality of health statuses at the different points in time.

18. The data analysis system of claim 14, wherein the user tools system combines the visualization with medical variables that relate to the health of the patient at the different points in time.

19. The data analysis system of claim 14, wherein the plurality of health statuses relate to a health of the patient's heart, and the health statuses are categorized as either compensated or decompensated.

20. A computer-implemented method for assessing the health of a patient's heart in a data processing system comprising a processing device and a memory comprising instructions which are executed by the processor, the method comprising:
collecting data from at least one data source, the at least one data source including past medical information for a particular patient, the past medical information comprising clinical narrative notes describing a health of the particular patient's heart;
performing natural language processing of the clinical narrative notes to extract information indicating the health of the patient's heart at different points in time;
categorizing the extracted information into either a compensated or a decompensated health status of the patient's heart at the different points in time; and
generating a user interface comprising the compensated or decompensated health statuses at the different points in time.
